(19) United States
(12) Patent Application Publication
(10) Pub. No.: US 2020/0222497 A1
(43) Pub. Date: Jul. 16, 2020

(54) USE OF COMPOSITAE-TYPE CYCLIC PEPTIDE COMPOUND AS CGAS-STING SIGNALLING PATHWAY INHIBITOR

(71) Applicant: CHINA PHARMACEUTICAL UNIVERSITY, Nanjing (CN)

(72) Inventors: Ninghua TAN, Nanjing (CN); Chen WANG, Nanjing (CN); Zhe WANG, Nanjing (CN); Senlin LI, Nanjing (CN); Huimin XU, Nanjing (CN); Lihua SONG, Nanjing (CN)

(21) Appl. No.: 16/639,736

(22) PCT Filed: Jan. 22, 2018
(86) PCT No.: PCT/CN2018/073571

§ 371 Date: Mar. 18, 2020

(30) Foreign Application Priority Data
Aug. 18, 2017 (CN) 201710710198.6

(51) Int. Cl. A61K 38/12; A61K 36/28

(52) U.S. Cl. CPC A61K 38/12; A61K 36/28

(57) ABSTRACT

The disclosure relates to application of a compositae type cyclopeptide compound or a pharmacologically permissible salt as a cGAS-STING signal channel inhibitor and application of the compositae type cyclopeptide compound or the pharmacologically permissible salt as the cGAS-STING signal channel inhibitor in preparing a drug for treating diseases caused by abnormal activation of a cGAS-STING signal channel. As the compositae type cyclopeptide compound is a natural compound diversified in dosage form and medication mode, the compositae type cyclopeptide compound has a wide clinical application prospect.

### TECHNICAL FIELD

The disclosure belongs to the drug technology, and in particular relates to application of a compositae type cyclopeptide as a cGAS-STING signal channel inhibitor and application thereof in preparing a drug for treating related diseases.

### BACKGROUND ART

Innate immunity is the first defensive line of the immune system of a host body. In the lengthy biological evolutionary process, a pattern recognition receptor in a host cell can recognize a pathogen-associated molecular pattern of a conservative constituent of invading pathogenic microorganisms, for example, a nucleic acid molecule and LPS. Once invasion of the pathogenic microorganisms is perceived, an infection signal is transferred by a downstream linker protein, a kinase and a transcription factor to induce expression of an I type interferon and a proinflammatory cell factor so as to eliminate the invading pathogenic microorganisms. A signal transduction mechanism that the host cell recognizes and eliminates non-self RNAs has been analyzed in detail in studies over the past few decades and a signal transduction mechanism of recognizing non-self DNAs, in particular the cGAS-STING signal channel, has been studied in recent years.

STING (stimulator of interferon genes) also called TMEM173, MITA, ERIS or MPYS as a intracellular key node molecule which responds DNA invasion responds to a signal from a cytoplasmic DNA receptor under stimulation of a cytoplasmic DNA, thereby playing a critical role in inducing a process of generating the interferon. The DNA recognition receptor of the host cell recognizing exogenous or endogenous non-self DNA transfers the signal to the node molecule STING on an endoplasmic reticulum and then the STING is dimerized quickly and is transferred from the endoplasmic reticulum to a peripheral nucleosome. In the process, the kinase TBK1 will also be recruited and transferred to the peripheral nucleosome to be activated, the activated TBK1 phosphorylates a transcription factor IRF3, and then the IRF3 is dimerized and enters into a cell nucleus to activate transcriptional expression of various target genes to take part in various biological effects including important physiopathologic processes such as virus resistance, inflammatory response, immune response, tumorigenesis and tumor progression.

The normally activated cGAS-STING signal channel helps the body to recognize and eliminate the invading DNA pathogenic microorganisms. But the abnormally excessively activated cGAS-STING signal channel will lead to inflammations and autoimmune diseases and the like of the body, for example, Aicardi-Goutieres syndrome, systemic lupus erythematosus or lupus-like diseases. Therefore, it is critically important to research regulation of the cGAS-STING signal channel to maintain innate immunity in a normal and stable state. Current studies are primarily focused on posttranslational modification of the key molecule of the cGAS-STING signal channel and search of new regulating molecules. There are relatively few studies in small molecular compounds taking part in regulating the cGAS-STING signal channel, but the studies have quite important application value, which is one of focused research directions.

In the prior art, reports on the cGAS-STING signal channel inhibitor are not available and reports on the compositae type cyclopeptide which acts on the channel and is used for preparing the drug for treating autoimmune diseases are not available, too. There are few types of the compositae type cyclopeptides in compositae plants, and only 15 compositae type cyclopeptides are separated from the Aster tataricus L. f. at present. It is of certain characteristics of extracting and separating the compositae type cyclopeptide compounds. It is difficult to obtain the cyclopeptide components which are relatively high in purity primarily because of small polarity, poor solubility and relatively low content of the compounds. In recent years, several methods of separating the cyclopeptide components from the Aster tataricus L. f. have been made public. For example, in a report by Xu Huimin et al., a series of cyclopeptides are obtained by the steps of performing reflux extraction by methanol on rhizomes of the compositae Aster tataricus L. f. to obtain a methanol extract; adding water to suspend; performing repeated extraction by ethyl acetate and normal butanol; performing forward and backward silica gel column chromatography and Sephadex LH-20 enrichment on the ethyl acetate part repeatedly to obtain a total cyclopeptide part; and separating and purifying the total cyclopeptide part in combination with HPLC. See Xu, H. M, et al. Astins K-P, six new chlorinated cyclopentapeptides from Aster tataricus, Tereahedron 2013, 69, 7964-7969. The patent CN102174083B also discloses a similar preparation method. But these methods commonly have the defects of low extraction efficiency, complex process, relatively large amount of loss of sample, poor repeatability and controllability and the like.

### SUMMARY OF THE INVENTION

The disclosure aims to provide application of a compositae type cyclopeptide compound as a cGAS-STING signal channel inhibitor and application thereof in preparing a drug for treating diseased caused by abnormal activation of a cGAS-STING signal channel.

In order to achieve the purpose, the disclosure provides the technical scheme as follows:

application of a compositae type cyclopeptide compound or a pharmacologically permissible salt as a cGAS-STING signal channel inhibitor; and

application of the compositae type cyclopeptide compound or the pharmacologically permissible salt as the cGAS-STING signal channel inhibitor in preparing a drug for treating diseases caused by abnormal activation of a cGAS-STING signal channel.

The compositae type cyclopeptide compound is preferably compositae type cyclopeptides Astins A-H (1-8) and Astins K-P (9-14) shown in the following structural formulae.

| 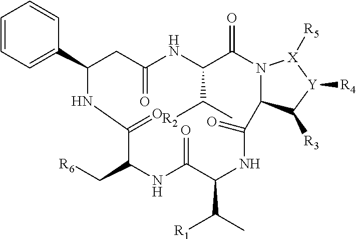 | | | | | | | |
|---|---|---|---|---|---|---|---|
| | | | | | | | 14 |
| 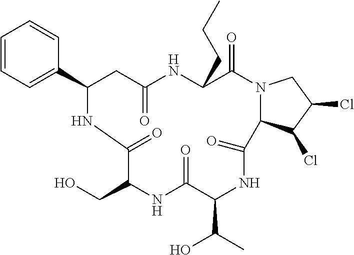 | | | | | | | |
| | R₁ | R₂ | R₃ | R₄ | R₅ | R₆ | X—Y |
| 1 | H | OH | Cl | Cl | H | OH | CH—CH |
| 2 | OH | H | Cl | Cl | Cl | OH | CH—CH |
| 3 | H | H | Cl | Cl | H | OH | CH—CH |
| 4 | H | H | H | H | Cl | OH | C═C |
| 5 | OH | H | H | H | Cl | OH | C═C |
| 6 | H | H | Cl | H | H | OH | CH—CH |
| 7 | H | H | H | H | H | OH | CH—CH |
| 8 | H | OH | H | H | Cl | OH | C═C |
| 9 | OH | OH | Cl | Cl | H | OH | CH—CH |
| 10 | OH | OH | H | H | Cl | OH | C═C |
| 11 | H | H | H | Cl | H | OH | CH—CH |
| 12 | H | H | H | Cl | H | OH | C═C |
| 13 | H | H | Cl | Cl | H | OAc | CH—CH |

Further preferably, the compositae type cyclopeptide is Astin C.

The compositae type cyclopeptide compound or the pharmacologically permissible salt can inhibit expression of downstream genes IFNβ, IFNα4 and CXCL10 of the cGAS-STING signal channel under stimulation of ISD, thereby inhibiting the cGAS-STING signal channel.

The pharmacologically permissible salt comprises a salt formed by an inorganic acid, an organic acid, an alkali metal, an alkaline-earth metal or an alkaline amino acid.

Further, the compositae type cyclopeptide or the pharmacologically permissible salt can be enumerated as a salt formed by the inorganic acid such as hydrochloric acid, nitric acid, sulfuric acid, phosphoric acid and hydrobromic acid, the organic acid such as maleic acid, fumaric acid, tartaric acid, lactic acid, citric acid, acetic acid, methanesulfonic acid, p-toluenesulfonic acid, hexanedioic acid, palmitic acid and tannic acid, the alkali metal such as lithium, sodium and potassium, the alkaline-earth metal such as calcium and magnesium, and the alkaline amino acid such as lysine.

The drug for treating the autoimmune diseases caused by abnormal activation of the cGAS-STING signal channel can be prepared by combining the compositae type cyclopeptide or the pharmacologically permissible salt thereof with a pharmaceutically acceptable carrier. Prepared drug dosage forms can be tablets, capsules, oral solutions, injections, lyophilized agents for injection or powder-injections and the like. As the compositae type cyclopeptide can be extracted and separated from aster, preparation of the drug dosage forms: tablets, capsules, oral solutions, injections, lyophilized agents for injection or powder-injections and the like is common acknowledge in the art, and therefore, various drug dosage forms prepared from the compositae type cyclopeptide compound and corresponding carriers can be implemented by those skilled in the art.

The pharmaceutically acceptable carriers above are conventional drug carriers in the pharmaceutical field, for example, a diluent and an excipient such as water, a filling agent such as starch and saccharose, an adhesive such as a cellulose derivative, alginate, gelatin and polyvinylpyrrolidone, a wetting agent such as glycerinum, a disintegrating agent such as agar, calcium carbonate and sodium hydrogen carbonate, an absorption promoter such as a quaternary ammonium compound, a surfactant such as hexadecanol, an adsorption carrier such as kaolin and bentonite clay and a lubricant such as talc powder, calcium stearate, magnesium stearate and polyethylene glycol. In addition, other auxiliary agents such as a flavoring agent and a sweetening agent can be also added into the composition.

The disclosed compound can be applied to patients needing the treatment by way of oral administration, nasal inhalation, rectal administration or parenteral administration. When the compound is used for oral administration, the compound can be prepared into conventional solid preparations such as tablets, powder, granules and capsules and can be prepared into liquid preparations such as a water or oil suspending agent or other liquid preparations such as syrup and elixir. When the compound is used for parenteral administration, the compound can be prepared into solutions for injection, a water or oil suspending agent and the like. Various dosage forms of the pharmaceutical composition can be prepared according to conventional production methods in the pharmaceutical field. For example, the active ingredient is mixed with one or more carriers and the mixture is prepared into needed dosage forms.

The drug preferably comprises 0.1-99.5wt % of active ingredient compositae type cyclopeptide or pharmacologically permissible salt thereof, most preferably 0.5-95wt % of active ingredient.

The application amount of the drug of the disclosure can be changed according to medication paths, ages and weights of patients, types and orders of severity of treated diseases and the like, and the daily dose of the drug can be 0.01-10 mg/kg weight, preferably 0.1-5 mg/kg weight. The drug can be applied at one time or at multiple times.

It is found that the compositae type cyclopeptide can inhibit the cGAS-STING signal channel in vivo and in vitro, is the first inhibitor of the channel and can be used for preparing drugs for treating related diseases by detecting influence of Astins A-H (1-8) and Astins (9-14) in an MEF cell on expression of a downstream gene of the cGAS-STING signal channel activated by simulation of the DNA analogue ISD and influence thereof on expression of an HSV-1 induced IFNβ protein in mouse serum. The diseases caused by abnormal activation of the cGAS-STING signal channel comprise autoimmune diseases, inflammatory diseases and cancers such as Aicardi-Goutieres syndrome and systemic lupus erythematosus.

As for the preparation method of the Astins A-H (1-8) and Astins (9-14), reference may be made to similar preparation methods of Xu, H. M, et al. Astins K-P, six new chlorinated cyclopentapeptides from Aster tataricus, Tereahedron 2013, 69, 7964-7969 and similar preparation method disclosed by the patent CN102174083B. But these methods commonly have the defects of low extraction efficiency, complex process, relatively large amount of loss of sample, poor repeatability and controllability and the like. The preparation method in the disclosure is improved and, to sum up, the improved preparation method comprises the step of performing silica gel column chromatography on methanol extract directly without extraction, so that the separating process is simplified and the sample loss is reduced. Part of pigments and other low polarity components can be removed simply by normal and reverse phase silica gel column chromatography. A part of small molecular components greatly different from the molecular weight of the cyclopeptide can be separated by means of Sephadex LH-20 gel chromatography, and meanwhile, the cyclopeptide is quickly enriched. The cyclopeptide can be separated successfully by means of a high performance liquid chromatography (HPLC) purification and recrystallization technique. In addition, only laboratory conventional chromatographic materials or industrial conventional chromatographic materials are used in the method, including normal and reverse phase silica gels, Sephadex LH-20 and the like. In a word, the extraction and separation method of the disclosure is good in controllability and repeatability, small in sample loss, relatively low in cost and convenient to operate, and micro cyclopeptide can be separated. A solvent can be recycled repeatedly, so that the method is suitable for industrial production. Specific preparation is seen in the examples.

The disclosure has the beneficial effects that the compositae type cyclopeptide compound disclosed by the disclosure for the first time is the cGAS-STING signal channel inhibitor which can inhibit expression of downstream genes IFNIβ, IFNα4 and CXCL10 of the cGAS-STING signal channel under stimulation of the DNA analogue ISD. Therefore, the compositae type cyclopeptide compound can be applied to preparing the drug for treating diseases caused by abnormal activation of the cGAS-STING signal channel. As the compositae type cyclopeptide compound is a natural compound diversified in dosage form and medication mode, the compositae type cyclopeptide compound has a wide clinical application prospect.

### BRIEF DESCRIPTION OF THE DRAWINGS

FIG. 1 is a flow diagram of a preparation method of a compositae type cyclopeptide compound of the disclosure;

FIG. 2a shows an inhibiting action of the compositae type cyclopeptide compound Astins A-H (1-8) and Astins K-P (9-14) in an MEF cell to expression of IFNβ mRNA activated by stimulation of a DNA analogue ISD;

FIG. 2b shows influence of Astin C (3) in the MEF cell on expression of downstream genes IFNβ, IFNα4 and CXCL10 of the cGAS-STING signal channel activated by stimulation of the DNA analogue ISD; and

FIG. 3 show influence of the compositae type cyclopeptide compound Astin C (3) on expression of IFNβ protein in HSV-1 induced mouse serum.

### DETAILED DESCRIPTION OF THE INVENTION

Substantial contents of the disclosure are further described below in combination with specific examples.

Unless otherwise specified, equipment, materials and reagents used in the examples can be purchased in the market.

### EXAMPLE 1

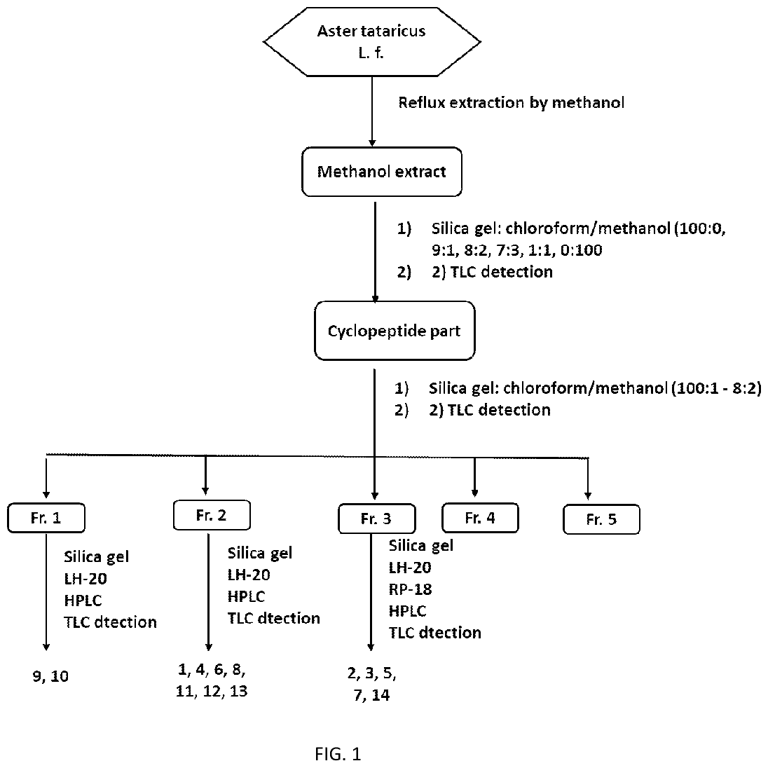

Preparation of compositae type cyclopeptide compounds Astins A-H (1-8) and Astins K-P (9-14) (the preparation method flow is shown in FIG. 1):

10 kg of dried roots and rhizomes of Aster tataricus L. was taken. The dried roots and rhizomes were dried, crushed and soaked in methanol overnight. Then heat reflux extraction by methanol was conducted for three times: 4 hours for the first time, 4 hours for the second time and 3 hours for the third time. Extraction liquids were combined. Concentration at a reduced pressure was conducted to obtain 4.6 kg of methanol extract. Silica gel column chromatography was conducted on the extract. Gradient elution was conducted by chloroform/methanol (100:0, 9:1, 8:2, 7:3, 1:1, 0:100). All fractions containing cyclopeptides were combined by means of a cyclopeptide TLC detection method to obtain a total cyclopeptide part (212.6 g). Separation and purification in the following processes were all guided in combination with the cyclopeptide TLC detection method. Silica gel column chromatography was conducted on the total cyclopeptide part. Gradient elution was conducted by chloroform/methanol (100:1 to 8:2). Combination was conducted to obtain five components Fr.1 to Fr.5 according to different cyclopeptide points. Silica gel column chromatography was conducted on Fr.1 (64 g). Gradient elution was conducted by petroleum ether/acetone (12:1 to 1:1) to separate, thereby obtaining three components Fr.1-1 to Fr.1-3. Sephadex LH-20 gel column chromatography was conducted on Fr.1-2 (10 g). Chloroform/methanol (1:1) was taken as an eluent. Silica gel column chromatography was conducted on the enriched cyclopeptide-containing part. Gradient elution was conducted by chloroform/ethyl acetate (10:1 to 3:1) to obtain three components Fr.1-2-1 to Fr.1-2-3. ODS HPLC semi-preparation column purification was conducted on Fr.1-2-1 (34 mg). 20% acetonitrile and 0.5% trifluoroacetic acid were taken as a moving phase. Astin L (10) (5 mg) was obtained. Silica gel column chromatography was conducted on Fr.1-2-2 (231 mg). Chloroform/ethyl acetate (1:3) was taken as an eluent. Astin K (9) (20 mg) was obtained. Silica gel column chromatography was conducted on Fr.2 (15 g). Gradient elution was conducted by petroleum ether/acetone (2:1 to 1:2) to obtain five components Fr.2-1 to Fr.2-5. Silica gel column chromatography was conducted on Fr.2-2 (2.8 g). Isocratic elution was conducted by chloroform/methanol (7:1) to obtain four components Fr.2-2-1 to Fr.2-2-4. Sephadex LH-20 chromatography was conducted on Fr.2-2-1 (786 mg). Chloroform/methanol (1:1) was taken as an eluent. A cyclopeptide component was obtained. ODS HPLC semi-preparation column purification was conducted on the cyclopeptide component. 35% acetonitrile and 0.5% trifluoroacetic acid were taken as a moving phase. Astin F (6) (10 mg), Astin H (8) (15 mg) and Astin M (11) (7 mg) were obtained. Sephadex LH-20 chromatography was conducted on Fr.2-3 (1.1 g). Chloroform/methanol (1:1) was taken as an eluent. A cyclopeptide component was obtained. Silica gel column chromatography was conducted on the cyclopeptide component. Gradient elution was conducted by chloroform/methanol (20:1 to 5:1) to obtain four components Fr.2-3-1 to Fr.2-3-4. ODS HPLC semi-preparation column purification was conducted on Fr.2-3-4 (41 mg) to obtain Astin N (12) (5 mg). Silica gel column chromatography was conducted on Fr.2-4 (789 mg). Gradient elution by chloroform/methanol (20:1 to 5:1) was conducted to obtain Astin D (4) (200 mg). Sephadex LH-20 chromatography was conducted on Fr.2-5 (971 mg). Chloroform/methanol (1:1) was taken as an eluent. Two components Fr.2-5-1 to Fr.2-5-2 were obtained. Silica gel column chromatography was conducted on Fr.2-5-1 (128 mg). Isocratic elution was conducted by ethyl acetate/methanol (25:1) to obtain Astin A (1) (12.7 mg). Repeated silica gel column chromatography was conducted on Fr.2-5-2 (78 mg). Elution and separation were conducted by chloroform/acetone to obtain Astin O (13) (3 mg). Silica gel column chromatography were conducted on Fr.3 (30 g). Gradient elution was conducted by petroleum ether/acetone (1:1 to 1:2) to obtain three components Fr.3-1 to Fr.3-3. Silica gel column chromatography was conducted on Fr.3-1 (8 g) and gradient elution was conducted by taking chloroform/ethyl acetate (3:1 to 5:1) as an eluent. Combination was conducted to obtain five components Fr.3-1-1 to Fr.3-1-5. Sephadex LH-20 chromatography was conducted on Fr.3-1-2 (3 g). Chloroform/methanol (1:1) was taken as an eluent. Cyclopeptide parts were combined. Methanol recrystallization was conducted on the part to obtain a component Astin C (3) (1.4 g). RP-18 column chromatography was conducted on Fr.3-2 (12 g). Gradient elution by 10-80% chloroform/water was conducted to obtain three components Fr.3-2-1 to Fr.3-2-3. Sephadex LH-20 chromatography was conducted on Fr.3-2-2 (4.1 g). Chloroform/methanol (1:1) was taken as an eluent. A cyclopeptide enriched part was obtained. Silica gel column chromatography was conducted on the part. Gradient elution was conducted by taking chloroform/methanol (20:1 to 5:1) as an eluent to obtain four components Fr.3-2-2-1 to Fr.3-2-2-4. ODS HPLC semi-preparation column purification was conduced on Fr.3-2-2-2 (821 mg). 45% acetonitrile and 0.5% trifluoroacetic acid were taken as an eluent. Astin P (14) (12 mg) and Astin E (5) (200 mg) were obtained. ODS HPLC semi-preparation column purification was conducted on Fr.3-2-2-3 (44 mg). 15% acetonitrile and 0.5% trifluoroacetic acid were taken as an eluent. Astin G(7) (12 mg) was obtained. Silica gel column chromatography was conducted on Fr.3-2-2-4 (1.3 g). Gradient elution was conducted by chloroform/ethyl acetate (1:3 to 1:9). The cyclopeptide part was enriched. Sephadex LH-20 chromatography and purification were conducted on the part. Then silica gel column chromatography was conducted on the part. Gradient elution was conducted by chloroform/methanol (15:1 to 5:1) to obtain a compound Astin B (2) (786 mg).

### EXAMPLE 2

Influence of the compositae type cyclopeptide compounds Astins A-H (1-8) and Astins K-P (9-14) prepared in example 1 on expression of downstream genes of the cGAS-STING signal channel activated by stimulation of the DNA analogue ISD in the MEF cell is detected. The experimental principle, the experimental method and the experimental result are as follows.

The experimental principle: In an innate immune signal transduction channel, the cGAS-STING signal channel can recognize exogenous DNA pathogenic microorganisms which invade the body. Exogenous DNA stimulation can induce expression of a downstream I type interferon gene and an interferon stimulated gene. Therefore, under stimulation of exogenous DNA (DNA analogue ISD), the activating condition of the cGAS-STING signal channel can be reflected by detecting expression of the downstream genes IFNβ, IFNα4 and CXCL10 of the channel, so that influence of the compounds on activation of the cGAS-STING signal channel is estimated.

The experimental method: (1) Cell culture: MEF cells were cultured by using DMEM (Invotrogen) containing 10% fetal calf serum (Gibco), 50 U/mL penicillin and 50 μg/mL streptomycin, with the culture condition of 37° C. and 5% CO2 and passaging was conducted once every two days. (2) Lipofection and compound treatment: The MEF cells were spread in a 12-pore cell culture plate. The cells were pre-treated for 6 hours by 10 μM corresponding compound and equal DMSO. 5 μg of ISD was used for transfecting so as to stimulate the cells by using Lipo2000 (Invotrogen). A culture solution was abandoned in 6 hours. The cells were centrifugally collected in an EP tube by PBS. (3) RNA extraction: The cells were lysed in (2) by 500 μL of TRIzol. Then 100 μL of CHCl₃ was added to extract RNA in lysate. Centrifugation was conducted for for 15 min at 12000 g at 4° C. 200 μL of uppermost supernatant was transferred to a new EP tube. Isometric isopropanol was added to precipitate the RNA. The RNA stood for 10 min. Then centrifugation was conducted for 10 min at 12000 g at 4° C. The supernatant was removed. 1 mL of DEPC water containing 75% ethanol to clean the precipitate. Centrifugation was conducted for 5 min at 7500 g at 4° C. The supernatant was removed. The precipitate was dried for 5 min at room temperature. Then RNA was dissolved with a proper amount of DEPC water for a follow-up experiment. (4) Expression of the downstream genes was detected by real time fluorescent quantitative PCR: Reverse transcription was conducted to obtain cDNA by taking the total RNA extracted from (2) as a template and oligo dT as a primer. The real time fluorescent quantitative PCR was performed by using a Power SYBR GREEN PCR MASTER MIX (ABI) reagent, and taking GADPH as an reference gene.

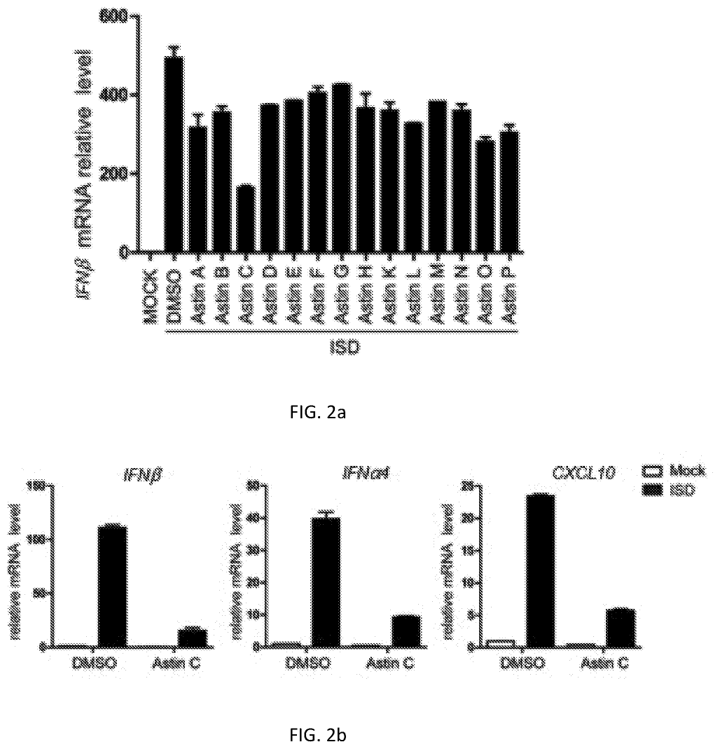

The experimental result is shown in FIG. 2. FIG. 2a shows an inhibiting action of the compositae type cyclopeptide compounds Astins A-H (1-8) and Astins K-P (9-14) in the MEF cell to expression of IFNβ mRNA activated by stimulation of the DNA analogue ISD. FIG. 2b shows influence of Astin C (3) in the MEF cell on expression of downstream genes IFNβ, IFNα4 and CXCL10 of the cGAS-STING signal channel activated by stimulation of the DNA analogue ISD.

The experimental result shows that the compositae type cyclopeptide compounds can inhibit expression of downstream genes IFNβ, IFNα4 and CXCL10 of the cGAS-STING signal channel under stimulation of the DNA analogue ISD. The activity of the Astin C is the best, which shows that the compositae type cyclopeptide can inhibit the activity of the cGAS-STING signal channel and is the first inhibitor of the channel that has been found.

### EXAMPLE 3

Influence of the compositae type cyclopeptide compound Astin C (3) prepared in example 1 on expression of IFNβ protein in HSV-1 induced mouse serum.

The experimental principle: Expression of the IFNβ protein in the body is a mark event of innate immunity resisting infection reaction, the expressed IFNβ can activate the signal transduction channel of the interferon receptor (IFNR) to induce expression of cell factors and inflammatory factors such as interferon stimulated genes, and finally, the invading pathogenic microorganisms are eliminated by recruiting NK cells or further activating molecular mechanisms such as adaptive immune response. Therefore, after the DNA virus HSV-1 infects a mouse, the activating condition of the innate anti-infection immune response of the body can be reflected by detecting the expression quantity of IFN-β of mouse serum, so that influence of the compound on the innate anti-infection immune reaction of the body is evaluated.

The experimental method: (1) Establishment of an HSV-1 infected mouse model: A 6-8 week old wild mouse was taken; 100 μL of HSV-1, the titer of which is 1×10⁷, was injected into a mouse body by way of tail vein injection; and serum was collected in 6 hours for a follow-up experiment. (2) The mouse was treated by the compound: The compound of certain concentration was injected into the wild mouse by way of tail vein injection for three times, once every another day. (3) Mouse blood was collected to obtain serum by an orbital blood taking method: The orbital blood capillary of the mouse was punctured by a capillary tube; 200 μL of peripheral blood was collected to an EP tube by drainage of the capillary tube; the blood stood overnight; then centrifugation was conducted for 10 min at 3000 rpm; and the supernatant was taken. (4) IFNβ protein content in serum was detected by ELISA: The IFNβ protein content in the mouse serum was detected according to a method of an experimental manual of a Verikine kit (PBL Assay Science).

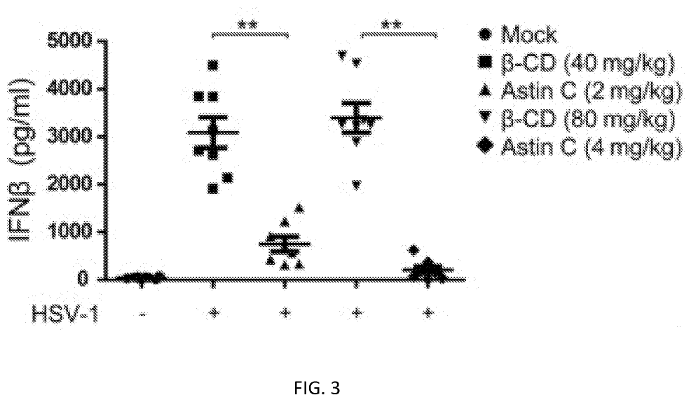

The experimental result is shown in FIG. 3.

The experimental result shows that the compositae type cyclopeptide compound Astin C (3) can reduce the HSV-1 induced IFNβ content in the mouse serum obviously, which shows that the compositae type cyclopeptide can inhibit the activity of the cGAS-STING signal channel in vivo.

### EXAMPLE 4

A 4% sulfuric acid ethanol solution was added into the compounds Astins A-H (1-8) and Astins K-P (9-14) in example 1 at PH=4; and filtering and drying were conducted to obtain sulfate compounds 1-14.

### EXAMPLE 5

A 4% hydrochloric acid solution was added into the compounds Astins A-H (1-8) and Astins K-P (9-14) in example 1 at PH=4; and filtering and drying were conducted to obtain hydrochlorate compounds 1-14.

### EXAMPLE 6

A 4% tartaric acid solution was added into the compounds Astins A-H (1-8) and Astins K-P (9-14) in example 1 at PH=4; and filtering and drying were conducted to obtain tartrate compounds 1-14.

### EXAMPLE 7

A 4% citric acid solution was added into the compounds Astins A-H (1-8) and Astins K-P (9-14) in example 1 at PH=4; and filtering and drying were conducted to obtain citrate compounds 1-14.

### EXAMPLE 8

Tablets: The tablets were prepared from the compounds Astins A-H (1-8) and Astins K-P (9-14) in example 1 or 10 mg of salts obtained in examples 4-7, 180 mg of lactose, 55 mg of starch and 5 mg of magnesium stearate.

Preparation method: The compounds or salts thereof were mixed with lactose and starch, the the mixture was uniformly wetted with water, the wetted mixture was screened and dried, then the mixture was screened, magnesium stearate was added, and then the mixture was tableted, wherein each tablet is 250 mg weight and the content of the compound is 10 mg.

### EXAMPLE 9

Agent in ampoule: The agent in an ampoule was prepared from the compounds Astins A-H (1-8) and Astins K-P (9-14) in example 1 or 2 mg of salts obtained in examples 4-7 and 10 mg of sodium chloride.

Preparation method: The compounds or salts thereof and sodium chloride were dissolved in a proper amount of water for injection; the solution was filtered; and an ampoule was filled with the solution in a sterile condition.

### EXAMPLE 10

Lyophilized agent for injection: The lyophilized agent for injection was prepared from the compounds Astins A-H (1-8) and Astins K-P (9-14) in example 1 or 10 mg of salts obtained in examples 4-7, 2 mg of sodium hydrogen carbonate and 252 mg of mannitol.

Preparation method: Water for injection was added to dissolve sodium hydrogen carbonate and mannitol; activated carbon was added to adsorb for 30 min to remove a heat source; filtering was conducted to remove the activated carbon; the compounds or salts thereof were added into the filtrate; ultrasonic treatment was conducted to dissolve the mixture; the PH was regulated to 5.0-7.0 by 1 N hydrochloric acid; the mixture was filtered by a microporous filter membrane; water for injection was added; and subpackaging, freezing and drying, plugging and capping were conducted.

### EXAMPLE 11

Capsules: The capsules were prepared from the compounds Astins A-H (1-8) and Astins K-P (9-14) in example 1 or 10 mg of salts obtained in examples 4-7, 187 mg of lactose and 3 mg of magnesium stearate.

Preparation method: The compounds or salts thereof were mixed with a cosolvent; the mixture was screened and uniformly mixed; and hard gelatin capsules were filled with the obtained mixture, wherein each capsule is 200 mg weight and the content of active ingredient is 10 mg.

What is claimed is:

1. A method for inhibiting cGAS-STING signal channel by adding a compositae type cyclopeptide compound or a pharmacologically permissible salt.

2. A method for treating a disease with abnormal activation of a cGAS-STING signal channel comprising a step administrating a subject in need for treating the disease with abnormal activation of a cGAS-STING signal channel with an effective amount of a compositae type cyclopeptide compound or a pharmacologically permissible salt.

3. The method according to claim 2, wherein the compositae type cyclopeptide compound is a monocyclic homogeneous pentapeptide compound which contains β-phenylalanine on a framework and is directly connected by normal amino acid peptide bonds, and the compositae type cyclopeptide compound comprises one L-β-phenylalanine, one L-serine derivative, one L-proline derivative and two other amino acid derivatives.

4. The method according to claim 3, wherein the compositae type cyclopeptide compound is compositae type cyclopeptides Astins A-H (1-8) and Astins K-P (9-14) shown in the following structural formulae:

| 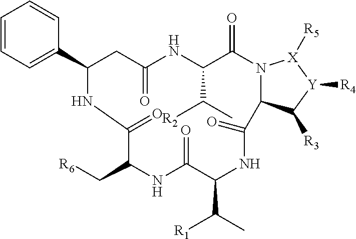 | | | | | | | |
|---|---|---|---|---|---|---|---|
| | | | | | | | 14 |
| 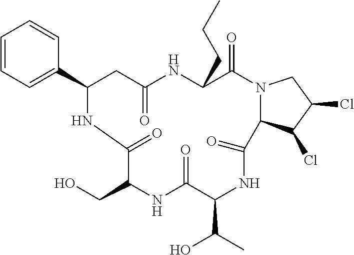 | | | | | | | |
| | R₁ | R₂ | R₃ | R₄ | R₅ | R₆ | X—Y |
| 1 | H | OH | Cl | Cl | H | OH | CH—CH |
| 2 | OH | H | Cl | Cl | Cl | OH | CH—CH |
| 3 | H | H | Cl | Cl | H | OH | CH—CH |
| 4 | H | H | H | H | Cl | OH | C═C |
| 5 | OH | H | H | H | Cl | OH | C═C |
| 6 | H | H | Cl | H | H | OH | CH—CH |
| 7 | H | H | H | H | H | OH | CH—CH |
| 8 | H | OH | H | H | Cl | OH | C═C |
| 9 | OH | OH | Cl | Cl | H | OH | CH—CH |
| 10 | OH | OH | H | H | Cl | OH | C═C |
| 11 | H | H | H | Cl | H | OH | CH—CH |
| 12 | H | H | H | Cl | H | OH | C═C |
| 13 | H | H | Cl | Cl | H | OAc | CH—CH |

5. The method according to claim 4, wherein the compositae type cyclopeptide is Astin C.

6. The method according to claim 2, wherein the compositae type cyclopeptide is extracted from roots and rhizomes of Aster tataricus L. f.

7. The method according to claim 1, wherein the compositae type cyclopeptide compound or the pharmacologically permissible salt is capable of inhibiting expression of downstream genes IFNβ, IFNα4 and CXCL10 of the cGAS-STING signal channel under stimulation of a DNA analogue ISD.

8. The method according to claim 1, wherein the pharmacologically permissible salt comprises a salt formed by an inorganic acid, an organic acid, an alkali metal, an alkaline-earth metal or an alkaline amino acid.

9. The method according to claim 2, wherein diseases caused by abnormal activation of the cGAS-STING signal channel comprise autoimmune diseases, inflammatory diseases and cancers.
